(19) United States
(12) Patent Application Publication
(10) Pub. No.: US 2004/0213758 A1
(43) Pub. Date: Oct. 28, 2004

(54) Hydroxyamate-containing materials for the inhibition of matrix metalloproteinases

(75) Inventors: Michael V. Sefton, Toronto (CA); Michael H. May, Brantford (CA); Gary A. Skarja, Toronto (CA)

Correspondence Address: RICHES, MCKENZIE & HERBERT, LLP, SUITE 1800, 2 BLOOR STREET EAST, TORONTO, ON, M4W 3J5, CA

(73) Assignee: Rimon Therapeutics Ltd.

(21) Appl. No.: 10/420,725

(22) Filed: Apr. 23, 2003

(51) Int. Cl. A61K 31/785

(52) U.S. Cl. 424/78.27

(57) ABSTRACT

Therapeutic polymers containing hydroxamate group that binds and thus inhibits zinc containing enzymes such as matrix metalloproteinases. The implantation of such material inhibits remodeling in its vicinity.

### FIELD OF THE INVENTION

This invention relates to therapeutic polymers containing a hydroxamate (HX) group that bind, and thus inhibit, zinc-containing enzymes, such as matrix metalloproteinases (MMPs). By inhibiting MMPs, the material, once implanted, inhibits tissue remodeling in its vicinity.

### BACKGROUND OF THE INVENTION

The following definitions and acronyms will be used in this specification:

| | |
|---|---|
| HX | hydroxamate |
| MMPs | matrix metalloproteinases or matrixins |
| TIMPs | tissue inhibitors of metalloproteinases |

Matrix metalloproteinases (MMPs), also called matrixins, are neutral zinc-dependent endopeptidases with substrate specificity for most extracellular matrix molecules, including collagens, gelatins, fibronectin, laminin and proteoglycan. To date, over 25 MMPs have been identified with many of them possessing a common name indicating the vulnerable extracellular matrix component: collagenases 1-4, gelatinases A-B, stromelysins 1-3, matrilysin, and enamelysin.

Cells do not constitutively express most MMPs in vivo; rather, growth factors, hormones, inflammatory cytokines, cell-matrix interactions and cellular transformation regulate their expression transcriptionally. Although the secretory granules of neutrophils and eosinophils are known to store MMP-8 and MMP-9, most cell types normally synthesize very low quantities of MMPs.

Extracellular matrix degradation is a normal event in the physiological remodeling associated with morphogenesis, reproduction, and in such growth and maintenance processes as cell migration, angiogenesis, and tissue regeneration. During inflammation and in several disease situations, however, excess MMPs degrade the surrounding proteinaceous matrix, which results in the destruction or weakening of connective tissue, unregulated cell migration/invasion, and tissue fibrosis. Inhibition of the activity of MMPs is one of the promising approaches for treating the medical disorders associated with elevated MMP levels.

Connective tissue weakening or destruction results in diseases such as rheumatoid arthritis, osteoarthritis, chronic periodontis, and arterial and cardiac aneurysm. MMP inhibitors have been used to treat osteoporosis, osteoarthritis, human chronic periodontal disease [Ashley, 1999; Reference 1] and various types of aneurysms [Thompson and Baxter, 1999; Reference 23, Prescott et al., 1999; Reference 18].

Chronic wounds take months or years to heal due, in part, to high levels of MMPs that degrade the newly formed matrix as fast as it is synthesized. The role of MMPs in the poor healing of gastric and skin ulcers [Trengrove et al, 1999; Reference 24, Saarialho-Kere, 1998; Reference 20] has been studied extensively. This work has not translated into significant research into the use of MMP inhibitors to treat chronic wounds [Parks et al., 1998; Reference 17], despite evidence that administration of GM6001, a collagenase inhibitor, increased the strength of linear incision rat skin wounds [Witte et al., 1999; Reference 29].

Angiogenesis or vasculogenesis of tumours and the formation of metastases require cell migration and invasion, which are enabled by the release of pro-MMPs. Various MMP inhibitors are being evaluated clinically for their anti-tumoral and antimetastatic potential [Drummond et al. 1999; Reference 4, Shalinsky et al., 1999; Reference 21]. Furthermore tissue remodeling occurs secondary to secretion or expression of MMP's. Thus blood vessels associated with wound repair are resorbed or ischemic tissue is destroyed by MMP action.

The activity of MMPs is essential for many of the processes involved in atherosclerotic plaque formation (infiltration of inflammatory cells, angiogenesis, and smooth muscle cell migration and proliferation). Elevated levels of MMPs are expressed in human atherosclerotic plaque and at the sites of aneurysm [Prescott et al., 1999; Reference 18]. Furthermore, matrix degradation by MMPs may cause the plaque instability and rupture that leads to the clinical symptoms of atherosclerosis. Recent studies using synthetic MMP inhibitors have highlighted the potential approach of MMP inhibition to treat atherosclerosis [George, 2000; Reference 8].

MMP activity is inhibited non-specifically by α₂-macroglobulin, a serum protein, and specifically in tissue by TIMPs, tissue inhibitors of metalloproteinases. The most popular approach to reducing MMP levels in tissue pharmacologically is the use of chelating agents such as antibiotics, tetracycline, thiols, carboxyalkyl, phosphonamidates and hydroxamates. These agents inactivate MMPs by binding the zinc at the active center of the enzymes. The hydroxamates are the most popular synthetic means of inhibiting MMP activity. With multiple point attachments, they behave like a molecular magnet for zinc.

Numerous soluble hydroxamates (e.g., Batimastat™, Marimastat™, Galardin™, Ro31-9790™) have been designed to broadly inhibit all MMPs, or inhibit one or more varieties of the same basic enzyme (e.g., the three collagenases) without any effect on related enzymes (e.g., stromelysin or gelatinase). The primary reason for making these inhibitors soluble is to enable systemic delivery. Modifications to the basic hydroxamate functionality have focused on reducing toxicity, increasing solubility, improving bioavailability, increasing stability and imparting specificity. Toxicity and specificity are concerns because MMPs play important roles in normal biological function and systemic delivery of broad-spectrum inhibition can interfere with their normal function. No consensus has yet been reached on whether MMP inhibitors should act on many MMPs or be highly specific. Typically, specificity is achieved by adding specific peptide sequences to molecules containing the hydroxamate group.

Currently, soluble hydroxamate compounds have been prepared with IC₅₀ between 1 and 5 nM for MMP-1, -3 and -7 [Chen et al., 1996; Reference 2]. Some hydroxamates such as Marimastat™ [Wojtowicz-Praga et al., 1996; Reference 33] and Trochate™ [Lewis et al., 1997; Reference 15]) are now in clinical trials.

The MMPs are a subclass of a larger (that is, greater than 200) set of proteases that depend on zinc for their catalytic activity. Some of these proteases have similar binding pockets as the MMPs, so it is possible that the inhibitors of MMPs may also inhibit the activity of other zinc proteases [Woessner, 1998; Reference 32].

Hydroxamate-containing polymers that are capable of reversibly binding a number of metal ions (e.g. V⁵⁺, Fe³⁺, Zn²⁺, Au³⁺, UO²⁺) have been proposed for use in several industrial and laboratory applications. These include the removal of metals from water [Vernon and Eccles, 1976; Reference 26], recovery of precious metals and metal catalysts in industrial processes [Vernon and Zin, 1981; Reference 25] and chromatographic separation [Kamble and Patkar, 1994; Reference 13]. As far as we can determine, no hydroxamate-containing polymers have been proposed to inhibit the activity of the Zn-containing MMPs. In fact, all known references to hydroxamate-containing polymers for biomedical applications deal with the chelation of iron or inhibition of nickel-containing urease. Applications include the treatment of iron overload from poisoning or transfusion-dependent anemias [Domb et al., 1992; Reference 7, Winston et al. 1985; Reference 30, Winston et al., 1986; Reference 31, Horowitz et al., 1985; Reference 10, Gehlbach et al, 1993; Reference 7], the coating of medical devices against coagulation [Domb et al., 1992; Reference 3], the in vivo inhibition of urease to reduce the incidence of infection-induced urinary stones [Domb et al., 1992; Reference 3], the widespread protection of tissues from iron-catalyzed oxygen free radical damage [Panter et al., 1992; Reference 16], protection from oxygen damage applied to the treatment of chronic wounds [Wenk et al., 2001; Reference 28], and the use of a hydroxamate-derivatized PEG as a renal magnetic resonance contrast agent [Duewell et al., 1991; Reference 5].

Two approaches have been employed to produce hydroxamate-containing polymers: 1) (co)polymerization of vinyl monomers bearing hydroxamate groups and 2) post-polymerization modification of polymer functional groups (e.g. carboxylic acid, ester, nitrile, amide) to generate hydroxamate groups.

Hydroxamate-bearing monomers were synthesized [Iskander et al. 2000; Reference 12] by reacting methacryloyl chloride (acid chloride of methacrylic acid) with hydroxylamine or various hydroxyalkyl hydroxamates under basic conditions. These monomers were then used to generate homo- and co-polymers by free radical polymerization processes. A number of researchers have generated hydroxamate-containing polymers via post-polymerization derivatization. Typically, the functionality is introduced via a nucleophilic displacement of polymer functional groups by hydroxylamine or hydroxylamine derivatives. Polymers derivatized in this way include polyacrylates [Kern and Schulz, 1957; Reference 14], polyacrylamide [Domb et al, 1992; Reference 3], polyacrylonitrile [Schouteden, 1958; Reference 19], and polyoxetanes [Xu et al, 1999; Reference 34]. Hydroxamate functionality was also imparted to polyethylene glycol [Duewell et al, 1991; Reference 5], various polysaccharides [Hallaway et al., 1989; Reference 9], and cellulose [Feldhoff, 1992; Reference 6] by activating hydroxyl groups for subsequent reaction with desferrioxamine-B, a tri-hydroxamic acid. Alternatively, polyacrylics may be directly reacted with hydroxylamine at high temperatures [Sparapany, 1989; Reference 22] or dehydrated to the corresponding anhydrides followed by reaction with hydroxylamine to generate hydroxamate functionality [Huffman, 1989; Reference 11].

### SUMMARY OF THE INVENTION

It is an object of the present invention to synthesize polymers containing HX groups which have the same biological effect as soluble hydroxamate MMP inhibitors, but that have many novel advantages. These materials, which combine the physiochemical properties of polymers with novel biological activity, are referred to as therapeutic polymers.

It is a further object of this invention to provide a novel polymer that inbibits the activity of biological species containing divalent metal ions, more specifically zinc-containing proteases and in particular, the matrix metalloproteinases, which are responsible for a variety of medical disorders when over-expressed.

It is still a further object of this invention to provide an MMP inhibitor that can be formed into various constructs and geometries, or incorporated into various medical devices.

It is a further object of this invention to provide a novel MMP inhibitor whose activity is localized to a specific tissue or site in the body. As a polymeric material, the inhibitor may remain insoluble or be formed in a way that restricts its movement or clearance from the site of application.

It is a still further object of this invention to provide an MMP inhibitor that has improved bioavailability for a specific dose and a desired length of time. Doses can be lower and administered less frequently because the inhibitor acts locally and persists locally. The duration of inhibition can be varied by changing the properties of the polymer (e.g., degradation, porosity, composition, geometry and size).

It is another object of this invention to provide a novel polymeric MMP inhibitor that is less toxic than the small, soluble MMP inhibitors. Systemic toxicity is reduced because the inhibitor acts locally. Local toxicity is reduced because lower dosages can be used, since clearance from the tissue is not significant. In addition, the inhibitor is a large M.W., insoluble synthetic polymer that cells cannot internalize or metabolize easily.

It is another object of this invention to provide an MMP inhibitor that is stable. This object is enabled by the fact that the inhibitor is an insoluble polymer, which is not degraded or metabolized easily by the body. In some situations a degradable HX polymer will be desirable, but in such cases, degradation can be controlled.

It is a further object of the invention to provide a novel method of removing MMPs in a safe and controlled manner. MMP-saturated constructs made from the non-degradable HX polymer can be removed by explantation or other means. A degradable version of the HX polymer would eventually become soluble and be cleared by the body after achieving its therapeutic purpose.

It is a further object of this invention to provide a method of derivatizing carboxylic-containing polymers to hydroxamic acid by a mixed anhydride intermediate (e.g., to make microbeads, nanoparticles and films).

It is a further object of this invention to provide a method of synthesizing a polymerizable hydroxamic acid unit by a mixed anhydride intermediate.

To this end, in one of its aspects, the invention provides a therapeutic polymer containing a hydroxamate group.

In another of its aspects, the invention provides a therapeutic polymer containing a hydroxamate group for binding zinc-containing enzymes.

In yet another of its aspects, the invention provides a medical device for the inhibition of matrix metalloproteinases which comprise a therapeutic polymer containing a hydroxamate group.

In still another of its aspects, the invention provides surface modified cross-linked polymethacrylic acid-co-methyl methacrylate beads containing a hydroxamate group.

A further aspect of the invention provides polymerizable therapeutic monomers containing a hydroxamate group.

In yet another of its aspects, the invention provides a hydroxamate group containing homopolymer.

A further aspect of the invention provides a hydroxamate group containing polymer synthesized by copolymerizing a polymerizable monomer containing a hydroxamate group with a comonomer.

A yet further aspect of the invention provides a matrix metalloproteinase inhibiting polymer containing a derivatizable polymer with a hydroxamate containing group grafted thereon.

In yet another of its aspects, the invention provides a method of derivatizing carboxylic-containing polymers to hydroxamic acid by a mixed anhydride intermediate.

A further object of the invention is to provide a method of synthesizing a polymerizable hydroxamic acid unit by a mixed anhydride intermediate.

A still further object of the invention is to provide therapeutic polymer for slowing, preventing or reversing tissue remodeling and destruction comprising a therapeutic polymer containing a hydroxamate group.

In yet another of its aspects, the invention provides therapeutic polymer for controlling inflammation comprising a therapeutic polymer containing a hydroxamate group.

A still further object of the invention is to provide beads for slowing, preventing or reversing tissue remodeling and destruction comprising a therapeutic polymer containing a hydroxamate group.

In yet another of its aspects, the invention provides beads for controlling inflammation comprising a therapeutic polymer containing a hydroxamate group.

In another of its aspects, the invention provides novel wound care products such as dressings, creams and ointments in which therapeutic polymers are incorporated.

A further aspect of this invention provides novel wound care products such as dressings, creams and ointments in which hydroxamate containing therapeutic polymers are incorporated.

In yet another of its aspects, the invention provides a thermoreversible gel in which hydroxamate beads are incorporated, which gel may be applied to a wound as a liquid and then removed by washing with cool saline.

In yet a further aspect, the invention provides a thermoreversible gel in which hydroxamate beads are incorporated, which thermoreversible gel comprises a copolymer and a solvent, the copolymer having the structure A(B)n, wherein A is soluble in the solvent, B is convertible between soluble and insoluble in the solvent depending on an environmental condition, and n is greater than 1, the gel being convertible from liquid to gel under an environmental condition wherein B is insoluble.

A further object of the invention is to provide a wound dressing which comprises a thermoreversible gel in which hydroxamate beads are suspended.

A yet further object of the invention is to provide a wound dressing which comprises a thermoreversible gel which comprises a copolymer and a solvent, the copolymer having the structure A(B)n, wherein A is soluble in the solvent, B is convertible between soluble and insoluble in the solvent depending on an environmental condition, and n is greater than 1, the gel being convertible from liquid to gel under an environmental condition wherein B is insoluble, in which hydroxamate beads are suspended.

### BRIEF DESCRIPTION OF THE DRAWINGS

FIG. 1 illustrates the chemical structure of the hydroxamate functional group.

FIG. 2 illustrates the inhibition of the degradation of gelatin tubes implanted in mice in the presence of hydroxamate-derivatized beads.

FIG. 3 illustrates the effects of the initial MAA content of cross-linked PMMA-MAA beads on the degree of hydroxamate derivatization.

FIG. 4 illustrates hydroxamate-derivatized beads with differing base surface MAA content stained with ferric chloride.

FIG. 5 illustrates fluorescence of FITC-labeled gelatin degradation products after incubation with MMP-2 and hydroxamate derivatized (right panel) versus underivatized (left panel) PMMA-MAA beads (63% initial MAA content).

FIG. 6 illustrates the NMR spectrum for hydroxamate monomer after purification to ˜95%.

### DETAILED DESCRIPTION OF THE INVENTION

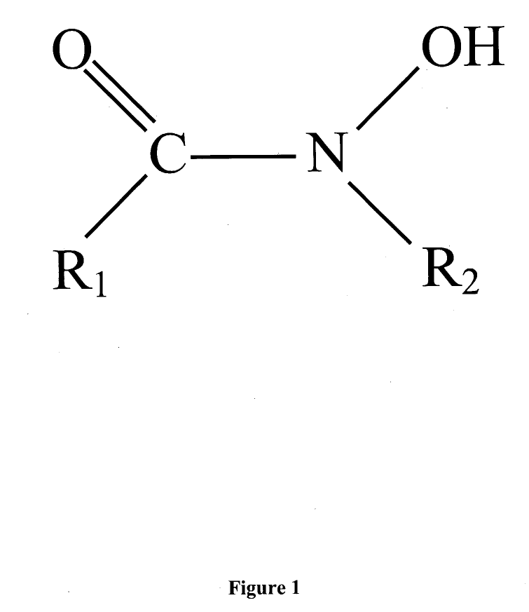

HX polymer is synthesized by surface modification of cross-linked polymethacrylic acid (PMAA)-co-methyl methacrylate (MAA) beads (resulting in a novel composition of PMAA-MMA-HX). In the example, with reference to FIG. 1, R1 represents the polymer main chain and R2 represents hydrogen. This method results in beads that are not soluble, but are useable as such; the surface modification method can be applied to other shapcs, but the materials will need to be in their final form prior to modification.

Polymerizable HX monomer was synthesized. This monomer can be used to synthesize an HX homopolymer or copolymerized with any other suitable comonomers to produce polymers with a variety of properties. These polymers are suitable for coating other materials (e.g., stainless steel) or ones made into a solid material after conventional thermoplastic processing (moulding, extrusion, etc.) or beads or nanoparticles made by spray drying, solvent evaporation or any other conventional polymer processing method. In the example, with reference to FIG. 1, R1 represents CH₂═C—CH₃ and R2 represents hydrogen.

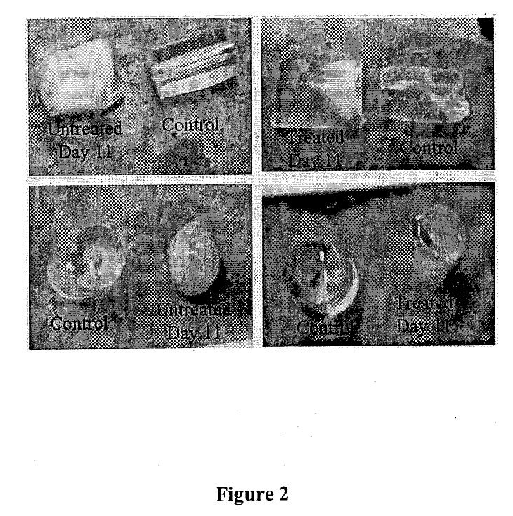

HX homopolymer synthesized from the HX monomer can also be grafted onto any derivatizable polymer to produce additional MMP-inhibiting polymers. In the example, with reference to FIG. 1, R1 represents any chemical group of a derivatizable polymer and R2 represents hydrogen. Small beads of HX polymer were injected in the vicinity of diseased or damaged tissue. Alternatively HX polymer can be incorporated into devices in contact with tissue. The incorporation of HX beads into the implant site of biomaterial tubes made from gelatin inhibited the remodeling and degradation of the gelatin tubes in a murine model. FIG. 2 illustrates the difference in degradation (at Day 11) of unimplanted (control) tubes versus tubes from untreated sites (no beads) and sites incorporating HX beads. The results showed that HX beads are capable of inhibiting tissue remodeling and destruction, controlling inflammation and restricting cell migration.

The hydrokamate beads may be incorporated into a thermoreversible gel that can be applied to a wound as a liquid and then removed by washing with cool saline. An example of such thermoreversible gel is disclosed in PCT published application serial number PCT/CA01/00325 (publication number WO 01/68768) filed on Mar. 15, 2001 in the name of Cheng and Lin, the specification of which is incorporated herein by reference. Thermoreversible gels undergo structural changes in response to changes in the environment. Within the composition, the copolymer undergoes a phase transition from liquid to gel in response to changes in an environmental parameter such as for example temperature, pH, ionic strength of the composition or combinations of these parameters.

The thermoreversible gel can be used as a protective coating for a wound. In this embodiment, the hydroxamate beads are incorporated into the gel itself, which is then applied to the wound as a liquid. The gel is then removed by washing with a cool saline. One example of a thermoreversible gel comprises a copolymer and a solvent, the copolymer having the structure A(B)n, wherein A is soluble in the solvent, B is convertible between soluble and insoluble in the solvent depending on an environmental condition, and n is greater than 1, the composition being convertible from liquid to gel under an environmental condition where B is insoluble. The environmental condition to conversion from liquid to gel may be temperature, pH, ionic strength and a combination thereof.

In the preferred structure of the gel, A is polyethylene glycol (PEG), polyvinyl pyrrolidone, polyvinyl alcohol, polyhydroxyethyimethacrylate, and hyaluronic acid, and B is poly-N-isopropyl acrylamide (PNIPAAm), hdroxypropylmethyl cellulose and other methyl cellulose derivatives, poly(thylene glycol vinyl ether-co-butyl vinyl ether), polymers of N-alky acrylamide derivatives, poly(amino acid)s, peptide sequences, poly(methacryloy L-alanine methyl ester), poly(methacryloy L-alanine ethyl ester) and nitrocellulose. The copolymer may be present in the solvent at a level from 5 to 50% by weight, preferably, from 10 to 25% by weight. Also, the integer n may represent 2, 4 or 8 with the preferred embodiment being greater or equal to 4.

In a specific preferred embodiment of the gel, the letter A represents polyethyleneglycol (PEG) and B represents poly-N-isopropyl acrylamide (PNIPPAAm) and the solvent is aqueous.

This gel may be formed by a process comprising the steps of: (i) forming a copolymer having the structure A(B)n, wherein A is soluble in a solvent of interest, B is convertible between soluble and insoluble in the solvent depending on an environmental condition, and n is greater than 1; (ii) solubilizing said copolymer in the solvent in an amount adequate to convert the composition from liquid to gel under an environmental condition where B is insoluble.

### EXAMPLES

### Example 1

### Surface Modification

Crosslinked poly(methyl methacrylate-co-methacrylic acid) (PMMA-MAA) beads were suspended in a suitable organic solvent (e.g. DMF, THF, diethyl ether) at approximately 10% wt/vol and allowed to equilibrate in solvent for at least 30 min at 0° C. while stirring. A 100% molar excess of N-methyl morpholine and chloroformate, relative to the MAA content of the beads, was added to the bead suspension. The reaction proceeded at 0° C. for 30 min. The beads were filtered from suspension and washed with DMF. The beads were transferred to a vessel containing a 100% molar excess of hydroxylamine solution in water and the reaction proceeded at ambient temperature for at least 1 hour. The beads were then filtered and washed with water, 0.1 M HCl, again with water, and then dried at 55-60° C.

FIG. 2 shows that the hydroxamate content (as indicated by nitrogen content) of the copolymer beads may be varied in this process by altering the acid content of the base copolymer from 15 to 80 mol % MAA.

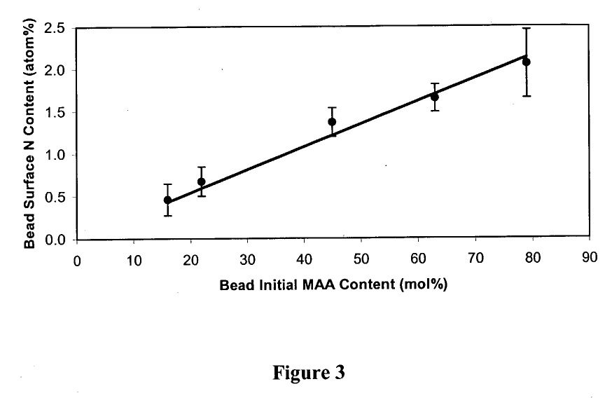

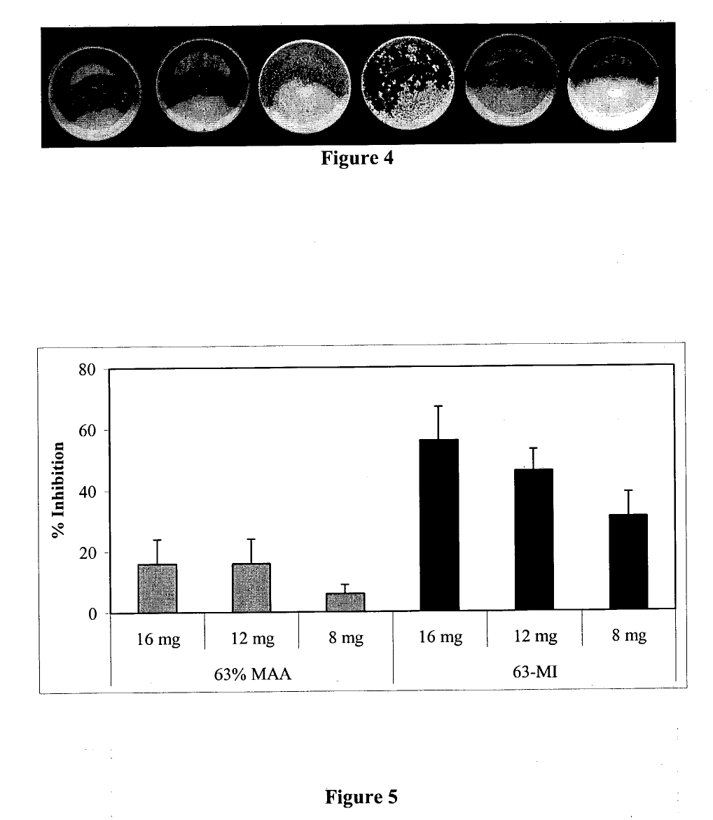

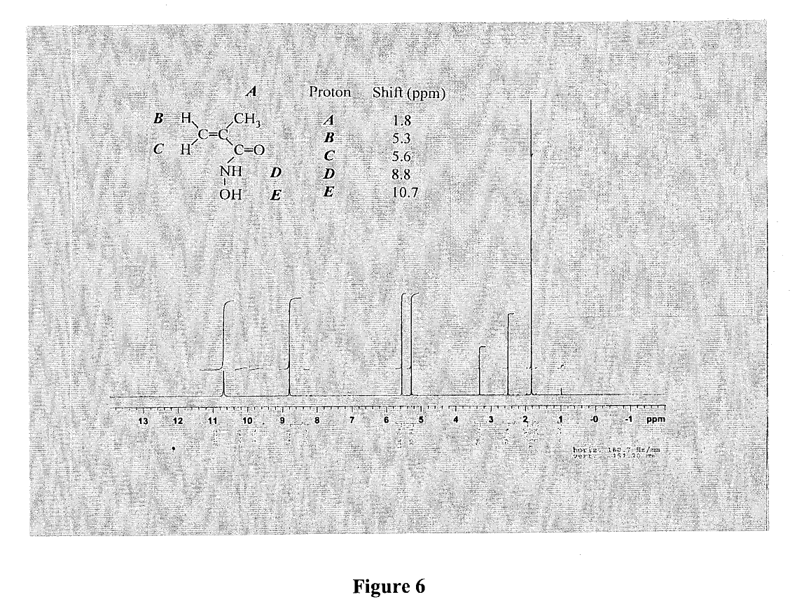

Ferric chloride stains hydroxamate groups with a purple colour. FIG. 3 shows the gradient in the staining of beads composed of a base polymer containing between 10 and 80% MAA that has been derivatized with hydroxamate groups, as well as the lack of staining for the underivatized 80% MAA beads (extreme right sample of beads in FIG. 3). The capacity of the hydroxamate-derivatized beads (from a 63% MAA base polymer) to inhibit the activity MMP-2 compared to underivatized beads is shown in FIG. 4. Before incubation with MMP-2 for 90 minutes at room temperature, HX and control beads were swollen in Tris-HCl/Ca buffer for 2 hours to eliminate any effects due to absorption. After pH adjustment with NaOH (to 7.6), the supernatant was incubated with FITC-gelatin for 60 minutes in the dark. MMP-2 activity was proportional to the intensity of solution fluorescence produced by the by-products of FITC-gelatin degradation.

### Example 2

### Bulk Modification

Polyacrylates may be derivatized via a nucleophilic displacement reaction by hydroxylamine in solution, yielding bulk modified, hydroxamate-containing copolymers. Poly(methylacrylate) was dissolved in DMF at approximately 10% wt/vol and the solution was placed in a sealed reactor and purged with dry, N₂ gas. The solution was heated to 45° C. and a 100% molar excess (relative to polymeric ester content) of hydroxylamine and 300% molar excess of N-methyl morpholine were added. The solution was stirred and the reaction was continued for 24 hr. The solution was cooled and the polymer was precipitated in a CaCl₂ solution. The polymer precipitate was then washed with 1 N HCl and deionized water before drying at 55° C.

### Example 3

### Hydroxamate Monomer Synthesis

Methacrylic acid monomer was dissolved in a suitable solvent (e.g. chloroform, diethyl ether) at 7% wt/vol and 0° C. A 25% molar excess of 4-methyl morpholine and 25% molar excess of chloroformate (relative to monomer carboxylic acid content) were added to the monomer solution with stirring. The reaction proceeded for 15 min. at 0° C., then the solution was filtered. The filtrate was added to a 25% molar excess of hydroxylamine in water solution and the combined solution was stirred at room temperature for 1 hr. After completion of the reaction, a solution of 0.05M NaOH was added to the reaction mixture. The aqueous layer was then separated from the organic phase and extracted three times with fresh organic solvent. The organic layer was extracted twice with 0.05 M NaOH and all of the aqueous volumes were combined. The aqueous raw monomer solution was dried in a freeze-dryer, leaving a white tacky solid. The raw product was then purified using silica gel chromatography (thin layer or column) with ethyl acetate/methanol or diethyl ether/methanol as the eluting solvent system. The column-purified monomer was then further purified by recrystallization from a 75/25 (vol/vol) toluene/chloroform solution to yield a colourless crystalline solid. Monomer purity was evaluated by NMR spectroscopy in d₆-DMSO (FIG. 5) and found to be approximately 95 mol %.

The ferric hydroxamate test was performed on the raw, derivatized monomer. The monomer was dissolved in 0.1 M HCl, several drops of 5 wt % FeCI₃ were added and the solution immediately turned dark burgundy confirming the presence of hydroxamate functionality. Performing the test on underivatized MAA resulted in no detectable colour change. In addition, the MMP inhibiting capacity of the purified monomer was demonstrated.

Although the invention describes and illustrates a preferred embodiment of the invention, it is to be understood that the invention is not so restricted and includes all alternative embodiments thereof.

### References

1. Ashley R. and the SDD Clinical Research Team (1999) Clinical trials of a matrix metalloproteinase inhibitor in human periodontal disease, Ann. New York Acad. Sci., 878, 159-79.

2. Chen, J. J., Y. P. Zhang, S. Hammond, N. Dewdney, T. Ho et al. (1996) Design, synthesis, activity & structure of a novel class of matrix metalloproteinase inhibitors containing a heterocyclic P-2′-P-3′ amide bond isostere. Biorg. Med. Chem. Lett., 123 6 1601-1606.

3. Domb et al. (1992) Method of making hydroxamic acid polymers from primary amide polymers, U.S. Pat. No. 5,128,420

4. Drummond A. et al. (1999) Preclinical and clinical studies of MMP inhibitors in cancer, Ann. New York Acad. Sci., 878, 159-79.

5. Duewell St. et al. (1991) Nonionicpolyethylene glycol-ferrioxamine as a renal magnetic contrast agent, Investigative Radiology, 26 (1), 50-57.

6. Feldhoff P. (1992) Comparison of coupling procedures for development of affinity membranes:optimization of the CDI method in Techniques in Protein ChemistryIII., Academic Press, London, 151-160.

7. Gehlbach P L, Purple R L, Hallaway P E, Hedlund B E (1993) Polymer conjugation reduces deferoxamine induced retinopathy in an albino rat model, Invest. Ophthalmol. Vis. Sci., 34(10), 2871.

8. George S. J. (2000) Therapeutic potential of matrix metalloproteinase inhibitors in atherosclerosis, Expert Opin Investig Drugs, 9:993-1007.

9. Hallaway P., Eaton J., Panter S., Hedlund B. (1989) Modulation of deferoxamine toxicity and clearance by covalent attachment to biocompatible polymers, Proc. Nat.'l Acad. Sci., 86 (24), 10108-12.

10. Horowitz, D., Margel S., Shimoni T., (1985) Iron detoxification by haemoperfusion through deferoxamine-conjugated agarose-polyacrolein microsphere beads, Biomaterials, 6, 9-16.

11. Huffman K. (1989) Carboxy hydroxamic acid polymers and their use as detergent additives, U.S. Pat. No. 4,874,539.

12. Iskander et al (2000) Synthesis and Copolymerization of methacryloyl hydroxamic acids, Journal of Applied Polymer Sciences, 78, 751-758.

13. Kamble K. and Patkar D. (1994) Synthesis and characterization of hydroxamic acid resin of poly(4-vinyl-2-carboxybenzophenone), Journal of Applied Polymer Science, 52, 1361-1364.

14. Kern W. and Schulz R. (1957) Synthetische makromolekulare stoffe mit reactiven gruppen, Angewandte chemie, 69 (5), 153-188.

15. Lewis, E. J., J. Bishop, K. M. K. Bottomley, D. Bradshaw, M. Brewster et al. (1997) Ro 32-3555, an orally active collagenase inhibitor, prevents cartilage breakdown in vitro and in vivo. Br. J. Pharmacol, 121, 540-546.

16. Panter S., Braughler J., Hall E. (1992) Dextran-coupled deferoxamine improves outcome in a murine model of head injury, Journal of Neurotrauma, 9 (1), 47-53.

17. Parks W., Sudbeck B., Doyle G. and Saariahlo-Kere U. (1998) Matrix metalloproteinases in tissue repair in Matrix Metalloproteinases, Editors: William C. parks and Robert P. Mecham, Academic Press, London, 263-297.

18. Prescott M. et al. (1999) Effect of matrix metalloproteinase inhibition on progression of atherosclerosis and aneurysm in LDL receptor-deficient mice over-expresing MMP-3, MMP-12, and MMP-13 and on restenosis in rats after balloon injury, Ann. New York Acad. Sci., 878, 159-79.

19. Schouteden F. (1958) On the conversion of amidoxime groups into hydroxamic acid groups in polyacrylamidoximes, Makromolekulare Chemie, 27(3), p. 246-255.

20. Saarialho-Kere U. (1998) Patterns of matrix metalloproteinase and TIMP expression in chronic ulcers, Arch. Dermatol. Res., 290, S47-S54.

21. Shalinsky D. et al. (1999) Broad antitumour and antiangiogenic activities of AG3340, a potent and selective MMP inhibitor undergoing advanced oncology clinical trials, Ann. New York Acad. Sci., 878, 159-79.

22. Sparapany J., Fong D., Fu J. (1989) Polyhydroxamic acid polymers from polyacrylic acids, U.S. Pat. No. 4,868,248.

23. Thompson R. and Baxter B. (1999) MMP inhibition in abdominal aortic aneurysms: rationale for a prospective randomized clinical trial, Ann. New York Acad. Sci., 878, 159-79.

24. Trengrove N. et al. (1999) Analysis of the acute and chronic wound environments: the role of proteases and their inhibitors, Wound Repair and regeneration, 7, 442-452.

25. Vernon F. and Zin W. (1981) Chelating ion exchangers containing N-substituted hydroxylamine functional groups Part 6. Sorption and separation of gold and silver by a polyhydroxamic acid, Analytica Chimica Acta., 123, 309-313.

26. Vernon F. and Eccles H. (1976) Chelating ion-exchangers containing N-substituted hydroxylamine functional groups Part III. Hydroxamic acids, Anal. Chim. Acta, 82.

27. Vernon F. and Eccles H. (1976) Chelating ion-exchangers containing N-substituted hydrokylamine functional groups Part IV. Column separations on a hydroxamic acid resin, Anal. Chim. Acta, 83.

28. Wenk J. et al. (2001) Selective pick-up of increased iron by deferoxamine-coupled cellulose abrogates the iron-driven induction of matrix-degrading metalloprteinase1 and lipid peroxidation in human dermal fibroblasts in vitro: a new dressing concept, The Journal of Investigative Dermatology, 116 (6), 833-839.

29. Witte M. et al. (1998) Metalloproteinase inhibitors and wound healing: a novel enhancer of wound strength, Surgery, 124 (2), 464-470.

30. Winston A., Varaprasad P., Metterville J., Rosenkrantz H. (1985) Evaluation of polymeric hydroxamic acid iron chelators for treatment of iron overload, Journal of Pharmacology and Experimental Therapeutics, 232 (3), 644-649.

31. Winston A., Varaprasad D. (1986) Polymeric iron chelators, U.S. Pat. No. 4,613,616.

32. Woessner Jr. J. (1998) The matrix metalloproteinase family in Matrix Metalloproteinases, Editors: William C. parks and Robert P. Mecham, Academic Press, London, 1-13.

33. Wojtowicz-Praga, S., J. Low, J. Marshall, E. Ness, R. Dickson et al. (1996) Phase 1 trial of a novel matrix metalloproteinase inhibitor batimastat (BB-94) in patients with advanced cancer. Invest. New Drugs 14:193-202.

34. Xu Y., Ogawa H., Kanoh S., Motoi M. (1999) Preparation and characterization of polyoxetane resins anchoring the pendant space-separated hydroxamic acid residue, Polymer Journal, 31, 143-149.

1. A therapeutic polymer containing a hydroxamate group.

2. A therapeutic polymer containing a hydroxamate group for binding zinc-containing enzymes.

3. A therapeutic polymer as claimed in claim 2 where said enzymes are matrix metalloproteinases.

4. Therapeutic polymers as claimed in claim 1 which inhibit the biological activity of species containing divalent metal ions.

5. A medical device for the inhibition of matrix metalloproteinases which comprise a therapeutic polymer containing a hydroxamate group.

6. A medical device as claimed in claim 5 wherein said polymer was synthesized by surface modification of cross-linked polymethacrylic acid-co-methyl methacrylate beads.

7. A surface modified derivatizable polymer as claimed in claim 1 containing a hydroxamate group.

8. The polymer of claim 7, wherein the derivatizable polymer is polymethacrylic acid-co-methyl methacrylate.

9. A polymerizable therapeutic monomer containing a hydroxamate group.

10. A hydroxamate group containing homopolymer.

11. A hydroxamate group containing polymer as claimed in claim 1 synthesized by copolymerizing a polymerizable monomer containing a hydroxamate group with a comonomer.

12. A therapeutic polymer as claimed in claim 1 containing a derivatizable polymer with a hydroxamate containing group grafted thereon.

13. The polymer of claim 12 wherein the graft consists of hydroxamate containing monomer units ranging 1 to 1,000,000 in number.

14. A method of derivatizing carboxylic-containing polymers to hydroxamic acid by a mixed anhydride intermediate.

15. A method of synthesizing a polymerizable hydroxamic acid unit by a mixed anhydride intermediate.

16. A therapeutic polymer as claimed in claim 1 for slowing, preventing or reversing tissue remodeling and destruction comprising a therapeutic polymer containing a hydroxamate group.

17. A therapeutic polymer as claimed in claim 1 for controlling inflammation comprising a therapeutic polymer containing a hydroxamate group.

18. A therapeutic polymer as claimed in claim 1 for restricting cell migration comprising a therapeutic polymer containing a hydroxamate group.

19. Beads for slowing, preventing or reversing tissue remodeling and destruction comprising a therapeutic polymer as claimed in claim 1 containing a hydroxamate group.

20. Beads for controlling inflammation comprising a therapeutic polymer as claimed in claim 1 containing a hydroxamate group.

21. Beads for restricting cell migration comprising a therapeutic polymer as claimed in claim 1 containing a hydroxamate group.

22. A wound care product which comprises a therapeutic polymer as claimed in claim 1 incorporated into a substrate.

23. A wound care product as claimed in claim 22 wherein said substrate is a dressing, a cream or an ointment.

24. A wound care product comprising a thermoreversible gel in which hydroxamate beads as claimed in claim 19 have been incorporated.

25. A wound care product as claim in claim 24 wherein said gelable composition comprises a copolymer and a solvent, the copolymer having the structure A(B)n, wherein A is soluble in the solvent, B is convertible between soluble and insoluble in the solvent depending on an environmental condition, and n is greater than 1, the composition being convertible from liquid to gel under an environmental condition where B is insoluble.

26. A wound care product as claimed in claim 25 wherein said environmental condition is selected from the group consisting of temperature, pH, ionic strength, and a combination thereof.

27. A wound care product as claimed in claim 25 wherein said environmental condition is temperature.

28. A wound care product as claimed in claim 25 wherein A is selected from the group consisting of polyethylene glycol (PEG), polyvinyl pyrrolidone, polyvinyl alcohol, polyhydroxyethylmethacrylate, and hyaluronic acid.

29. A wound care product as claimed in claim 25 wherein B is selected from the group consisting of poly-N-isopropyl acrylamide (PNIPAAm), hydroxypropylmethyl cellulose and other methyl cellulose derivatives, poly(ethylene glycol vinyl ether-co-butyl vinyl ether), polymers of N-alky acrylamide derivatives, poly(amino acid)s, peptide sequences, poly(methacryloy L-alinine methyl ester), poly(methacryloy L-alanine ethyl ester) and nitrocellulose.

30. A wound care product as claimed in claim 25 wherein the copolymer is present in the solvent at a level of from 5% to 50% by weight.

31. A wound care product as claimed in claim 25 wherein the copolymer is present in the solvent at a level of from 10% to 25% by weight.

32. A wound care product as claimed in claim 25 wherein n is 2, 4 or 8.

33. A wound care product as claimed in claim 25 wherein n is greater than or equal to 4.

34. A wound care product as claimed in claim 25 wherein A is polyethyleneglycol (PEG).

35. A wound care product as claimed in claim 25 wherein B is poly-N-isopropyl acrylamide (PNIPAAm).
